(19) United States
(12) Patent Application Publication
(10) Pub. No.: US 2024/0399130 A1
(43) Pub. Date: Dec. 5, 2024

(54) FLEXIBLE WEARABLE TRANSDERMAL DRUG DELIVERY DEVICE

(71) Applicant: The Chinese University of Hong Kong, Hong Kong (CN)

(72) Inventors: Ni ZHAO, Hong Kong (CN); Jiangtao LIU, Baoding (CN); Cunman LIANG, Tangshan (CN); Yixin QI, Hangzhou (CN)

(21) Appl. No.: 18/678,495

(22) Filed: May 30, 2024

Related U.S. Application Data
(60) Provisional application No. 63/504,814, filed on May 30, 2023.

(51) Int. Cl. A61M 37/00; A61N 1/04; A61N 1/36

(52) U.S. Cl. CPC A61M 37/0092; A61N 1/0428; A61N 1/36031; A61M 2037/0007; A61M 2205/055; A61M 2205/3317; A61M 2205/50; A61M 2230/65

(57) ABSTRACT

The subject invention pertains to a novel design and fabrication method for a transdermal drug delivery (TDD) device with one or both of ultrasonic and electrical stimulations to enhance drug penetrating through skin surface and a skin impedance sensor to enable closed-loop control providing stable drug release through the skin. The invention also provides a layered layout of the device and a corresponding fabrication protocol for realizing monolithic integration of the stimulation and sensing components and robust contact of the TDD device with skin. A flexible and layered architecture is provided to improve manufacturability, effectiveness, compactness, and usability of the provided TDD in clinical applications.

### CROSS-REFERENCE TO RELATED APPLICATION

This application claims the benefit of U.S. Provisional Patent Application Ser. No. 63/504,814, filed May 30, 2023, which is hereby incorporated by reference in its entirety including any tables, figures, or drawings.

### BACKGROUND OF THE INVENTION

Existing transdermal drug delivery (TDD) devices rely on either passive structures such as micro-needles, which offer poor control over drug penetration rate, or active devices, such as iontophoresis, which often suffer from low safety, low efficiency, or large device size. Furthermore, none of the related art devices have sensing modules to provide feedback on skin condition to allow for closed-loop control of accurate and stable drug release.

### BRIEF SUMMARY OF THE INVENTION

Embodiments of the subject invention provide compact, flexible, and low-voltage TDD devices with high drug delivery efficiency and precise (e.g., patient-specific) control over drug dose. Embodiments of the provided TDD devices outperform related art technologies in performance, accessibility, and safety.

Embodiments provide a design and fabrication protocol of a TDD device with one or both of ultrasonic and electrical stimulations advantageously configured and adapted (a) to enhance drug penetration through the skin surface and (b) with an impedance sensor to help control stable closed-loop drug release or dosing. In certain embodiments, to make robust contact with the skin, a novel fabrication method for a flexible layered structure is provided. Embodiments of this feedback-controlled multi-modality system with flexible device layout can provide high efficiency and stable transdermal drug delivery.

FIG. 1 illustrates an exemplary embodiment of the layout of the provided TDD device, where an ultrasonic generator (e.g., ultrasonic transducer) layer (170), an iontophoresis electrode layer (140) and a sensor electrodes layer (120) comprising a pair of thin conductive flexible electrodes are aligned in the layered concentric substrates. In certain embodiments an impedance sensor positioned on or in the skin friendly contact layer (110) can provide close contact with skin and can monitor skin status continuously, and the result can be used to evaluate drug release efficacy. Skin impedance is a critical physical parameter that indicates diffusion coefficient of the skin, and it can be measured by the signal collection system (A) in FIG. 1A. In certain embodiments, there are two controllers (B) and (C) for generating electrical and ultrasonic stimulations respectively. The provided wearable transdermal drug delivery device with an ultrasonic or electric stimulation or both can be precisely controlled. One critical issue for performance of such a transdermal drug delivery device is that, when using the ultrasonic/electrical dual modality stimulation, the ultrasonic stimulated skin region should be largely overlapped with the skin region exposed under the external electric field. The provided layered structure with flexible substrates not only can arrange proper overlapping position, alignment, and control for both ultrasonic and electric stimulations, but also can allow the device to contact closely with skin. Accordingly, this embodiment helps achieve self-administration of transdermal drugs with controlled release.

Embodiments provide a control unit (520) comprising a signal collector, an iontophoresis controller, and an ultrasonic generation controller; a sensing layer (101) operably connected to the control unit (520), the sensing layer comprising: a skin friendly contact layer (110), and a sensor electrodes layer (120); a multi-modality stimulation layer (105) physically connected directly on the sensing layer, the multi-modality stimulation layer comprising: an electrical stimulation layer (102) operably connected to the control unit (520), the electrical stimulation layer (102) comprising an iontophoresis electrode layer (140), and a substrate layer for electrodes (150); and an ultrasonic stimulation layer (103) operably connected to the control unit (520) and physically connected directly on the electrical stimulation layer (102), the ultrasonic stimulation layer (103) comprising: an ultrasonic generator layer (170), and a bottom substrate layer with ultrasonic connectors (190).

By layer is meant a collection of elements, structures, or features with a specified level of commonality in design, construction, purpose, or function. A layer can have sub-layers, or can itself be a sub-layer, unless otherwise specified. A layer can be connected to, provided on, separated from, or provided directly on another layer or another feature or design element. For example, in certain embodiments, sensing layer (101) can comprise a skin friendly contact layer (110), and a sensor electrodes layer (120); a multi-modality stimulation layer (105) can be provided directly on sensing layer (101) and can comprise an electrical stimulation layer (102) and an ultrasonic stimulation layer (103), each, respectively, operably connected to a control unit (520); the electrical stimulation layer (102) comprising an iontophoresis electrode layer (140), and a substrate layer for electrodes (150); and the ultrasonic stimulation layer (103) comprising: an ultrasonic generator layer (170), and a bottom substrate layer with ultrasonic connectors (190). An electrical stimulation layer (102) and an ultrasonic stimulation layer (103), each, respectively, can be provided on (e.g., separated only by one or more of an insulating, adhesive, or barrier layer) or directly on (e.g., in direct contact with or fastened directly to) the other, or can be provided on (e.g., separated only by one or more of an insulating, adhesive, or barrier layer) or directly on (e.g., in direct contact with or fastened directly to) another layer, such as a sensing layer (101) or other functional layer.

Additional embodiments provide other designs of ultrasonic element geometry (e.g., as shown in FIG. 1A (right side), and FIG. 1B), which illustrate a ring type ultrasonic transducer and an ultrasonic transducer array. The ring type ultrasonic transducer is designed with a ring electrode used as ultrasonic stimulation generator, in contrast to the solid disc ultrasonic element geometry (e.g., as shown in FIG. 1 (left side)). The ultrasonic transducer array is designed with a corresponding number of stimulating electrodes and a common ground electrode used as ultrasonic stimulation generator, so that each ultrasonic transducer is powered individually to focus and scan the ultrasonic wave beams, resulting in high ultrasonic power and large ultrasonic action area with a low local voltage.

### BRIEF DESCRIPTION OF THE DRAWINGS

FIGS. 1A and 1B illustrate a layout of a TDD device (100) comparing two types of ultrasonic elements (160, 170, 180, 190) (i.e., solid piece elements on the left side; ring type elements on the right side; and an ultrasonic transducer array in FIG. 1B), each respectively according to an embodiment of the subject invention. From top to bottom, the names of the provided layers are skin friendly contact layer (110), sensor electrodes layer (120), a polymeric flexible layer (130) (e.g., used as the drug reservoir and as a substrate of the sensor electrodes)), iontophoresis electrode layer (140), substrate layer for electrodes (150), top surface layer of ultrasonic unit (160), ultrasonic generator layer (170), supplemental material (e.g., for enhancing the ultrasonic generator) layer (180), and bottom substrate layer with ultrasonic connectors (190). The controlling parts contain three blocks (schematically illustrated on the left side image only, but contemplated for use in the right side of FIG. 1A, as well as in other embodiments), signal collection unit (A), iontophoresis controller (B), and ultrasonic generation controller (C), each respectively, operably connected as shown and physically located either on the TDD or optionally remote from the TDD.

FIG. 2 illustrates a sensor unit of a TDD device (200) according to an embodiment of the subject invention, showing (left side) overall device structure of a three electrode impedance measurement system, and (right side) layout components of a measurement system comprising skin friendly contact layer (110), sensor electrodes layer (120), and a polymeric flexible layer (130).

FIG. 3 illustrates a fabrication flow of the electrical stimulation unit (300) of a transdermal drug delivery device according to an embodiment of the subject invention, showing (left side) a schematic illustration of fabrication steps and materials, and (right side) a prototype device structure and layers schematic.

FIG. 4 illustrates a fabrication process of an ultrasonic unit (400) according to an embodiment of the subject invention, the process comprising (410) spin coating of poly(methyl methacrylate) (PMMA) and Polyimide (PI); (420) deposition of gold electrode; (430) PZT element connected to substrate; (440) sealing of PZT element using polydimethylsiloxane (PDMS), and (450) dissolving the sacrificial layers allowing peeling-off of the unit from silicon wafers.

FIG. 5 illustrates a functional block diagram of a device (500) according to an embodiment of the subject invention.

FIG. 6 illustrates a physical assembly schematic diagram of a device (600) according to an embodiment of the subject invention.

FIG. 7 illustrates a physical device (600) positioned on a dermal layer according to an embodiment of the subject invention.

FIGS. 8A-8E illustrate the design, execution, and results of a finite element simulation of drug penetration through the skin structure, according to an embodiment of the subject invention, including schematic (FIG. 8A) and graphical (FIG. 8B) model representations, and simulation results for drug penetration under three simulation conditions, i.e., (FIG. 8C) passive diffusion, (FIG. 8D) iontophoresis only, and (FIG. 8E) ultrasonic-enhanced iontophoresis.

FIG. 9 illustrates the drug penetration rate by the “iontophoresis only” and “ultrasonic-enhanced iontophoresis” devices according to respective embodiments of the subject invention, as compared to a “passive” penetration control.

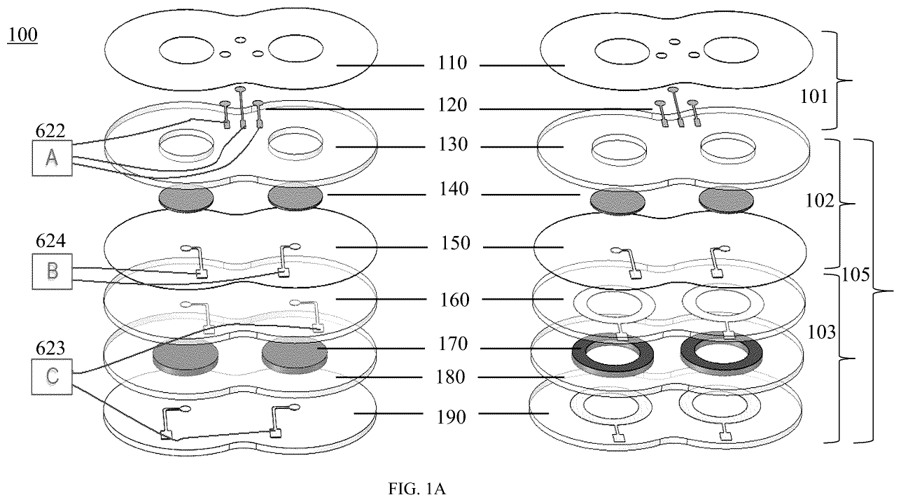

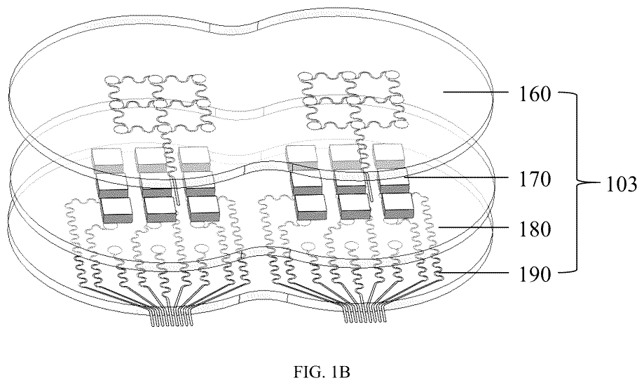

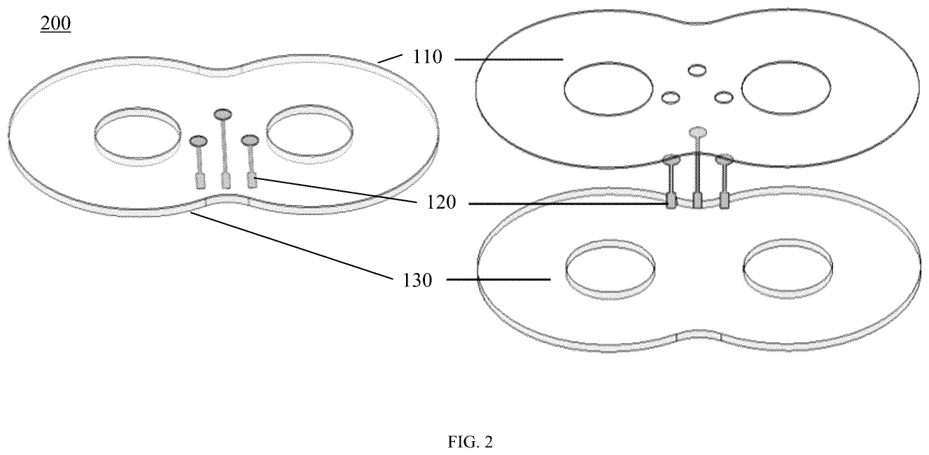

### DETAILED DISCLOSURE OF THE INVENTION

In certain embodiments of the subject invention, the provided device can be divided into two parts, with (1) a sensor unit assembled on the top (e.g., at the surface right next to the skin), and (2) a set of stimulation units, such as ultrasonic devices and iontophoresis electrodes, assembled at the bottom (e.g., at a surface away from the skin). The device can be assembled in an orientation such that the top, or last surface to be assembled, can be the first surface to contact the skin. The fabricated layers of the provided device from top to bottom can comprise:
Layer 1: A skin friendly contact layer (110) (e.g., an elastomer or polymeric layer for non-damaging interface with the skin, and/or for encapsulation of the device)
Layer 2: Sensor electrodes layer (120) (e.g., for placing sensors in contact with or in proximity to the skin)
Layer 3: A polymeric flexible layer (130) (e.g., used as the drug reservoir and as a substrate of the sensor electrodes)
Layer 4: An iontophoresis electrode layer (140) (e.g., for electrical stimulation)
Layer 5: A substrate layer (150) (e.g., with deposited gold lines)
Layer 6: A top surface layer (160) (e.g., a substrate for the ultrasonic anode electrode)
Layer 7: An ultrasonic generator layer (170) (e.g., for driving the ultrasonic)
Layer 8: A supplemental material layer (180) (e.g., for improving ultrasonic function)
Layer 9: A bottom substrate layer (190) (e.g., including the ultrasonic electrode connectors)

In certain embodiments Layers 1-2 can form a sensing layer, Layers 3-5 can form an electrical stimulation unit, and Layers 6-9 can form an ultrasonic stimulation unit. The provided layer-by-layer fabrication method advantageously enables the device to work at single stimulation mode (e.g., by effectively skipping, omitting, or deactivating layers 3-5 or layers 6-9, respectively, either in fabrication or in operation), dual stimulation mode, or even multiple stimulation modes with additional functional layers (e.g., heating, cooling, monitoring, storage, delivery, or stimulating layers) inserted and/or activated in the device.

Embodiments provide a sensing layer providing skin impedance measurement and other functions or alternative electrode designs. The skin impedance value is a critical parameter for assessing the skin permeability. In certain embodiments, skin impedance can provide a feedback on skin condition after the stimulation and can be useful as a reference for adjusting the stimulation intensity (e.g., the strength of an ultrasonic or electric field.) The sensor can be a simple three-electrode system and the electrodes can be made from Ag/AgCl, metal, conductive polymer, conductive hydrogel, or other conductive materials; for conductive polymers, some additives (e.g., dopamine, (3-glycidyloxypropyl) trimethoxysilane (GOPS)) can improve the conductivity, working time and mechanical stability of electrodes.

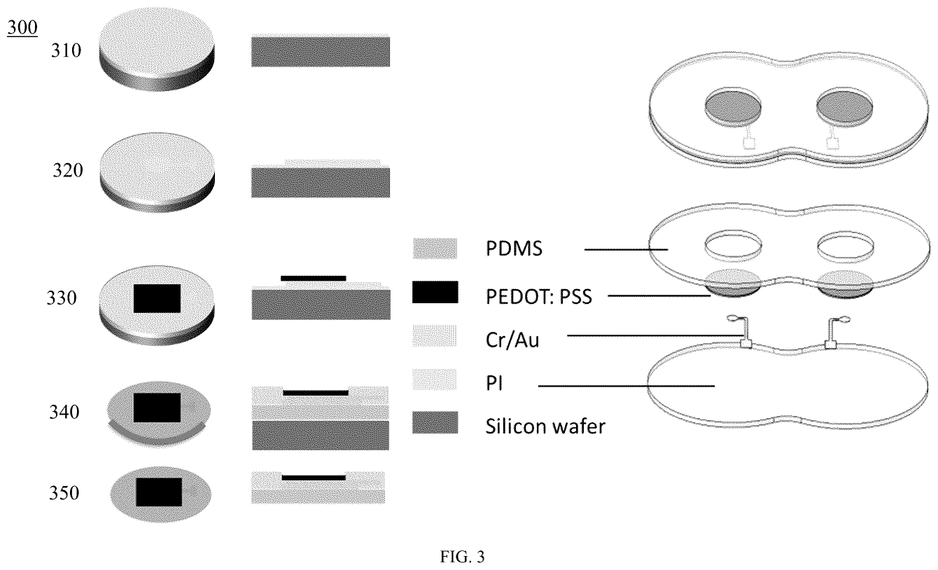

FIG. 3 presents a general fabrication process of an electrical stimulation unit (300) according to an embodiment of the subject invention, which comprises the following steps:
(310) deposition of a lift-off (e.g., PI in the figure) layer on a silicon wafer;
(320) deposition of a metal contact on the lift-off layer;
(330) deposition of an electrode material;
(340) encapsulation with a sealing layer (i.e., PDMS in the figure); and
(350) peel-off of the electrode from the silicon wafer. (This step can be beneficially enhanced or enabled by the weak interaction between the lift-off layer and the silicon wafer in certain embodiments.)

In certain embodiments the embedded iontophoresis electrodes can be made of any biocompatible ion conductive materials including but not limited to poly(3,4-ethylenedioxythiophene): polystyrene sulfonate (PEDOT:PSS), polyaniline (PANI), conductive hydrogel, and other suitable materials as known in the art or as may be later developed, and for conductive polymers, some additives (e.g., dopamine, (3-glycidyloxypropyl) trimethoxysilane (GOPS)) can improve the conductivity, working time and mechanical stability of electrodes. The scaling layer can be made of Eco-flex, PDMS, rubber or other suitable (e.g., curable) materials. The sealing layer is typically achieved by depositing uncured liquids (e.g., PDMS precursor solution) to the substrate, followed by a standard curing process. In certain embodiments, the sealing layer material can be soft and stretchable to advantageously allow for close contact with the skin.

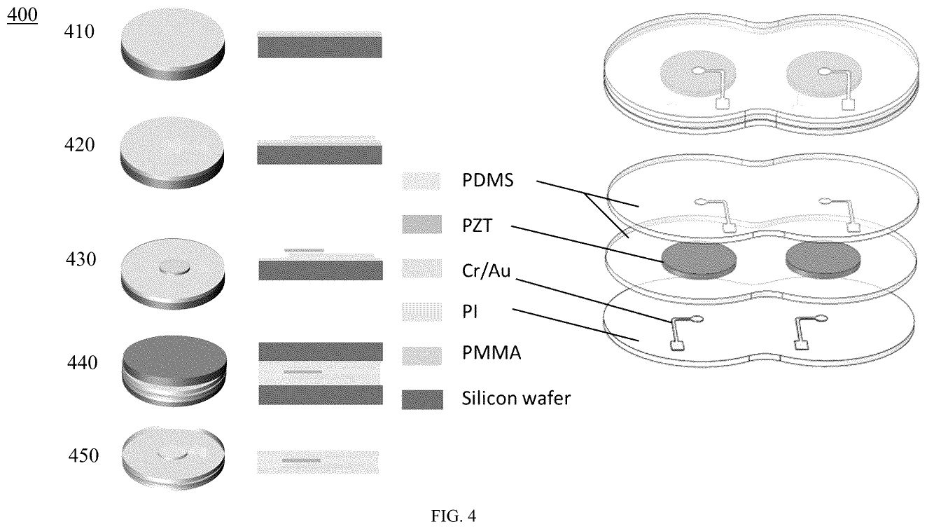

FIG. 4 illustrates a fabrication flow of an ultrasonic unit (400) according to an embodiment of the subject invention. First a silicon wafer is cleaned and dried. A sacrificial layer (e.g., typically made of PMMA) is first deposited on the silicon wafer and briefly thermally annealed, followed by deposition of a PI supporting layer (410). After depositing bottom metal contact (420), the ultrasonic transducer was bonded (e.g., PZT material) (430). To finish the unit, another piece of silicon wafer coated with a PMMA sacrificial layer and PI coated with a top metal contact is prepared; then two wafers are stacked face-to-face with each respective ultrasonic transducer from each respective device aligned face-to-face and sandwiched between the wafers (440). PDMS or other curable epoxy is then applied to fill the gaps around the ultrasonic device and bind the device to the sealing layer. Finally, the sacrificial layers are dissolved by a solvent (e.g., acetone for PMMA), allowing for peeling-off of the ultrasonic unit from both silicon substrates (450).

In certain embodiments the embedded ultrasonic transducer can be made of any piezoelectric materials including, but not limited to, Lead zirconate titanate (PZT), Pb(Mg_{1/3}Nb_{2/3})O₃—PbTiO₃ (PMN-PT) ceramics, and other suitable materials as known in the art or as may be later developed.

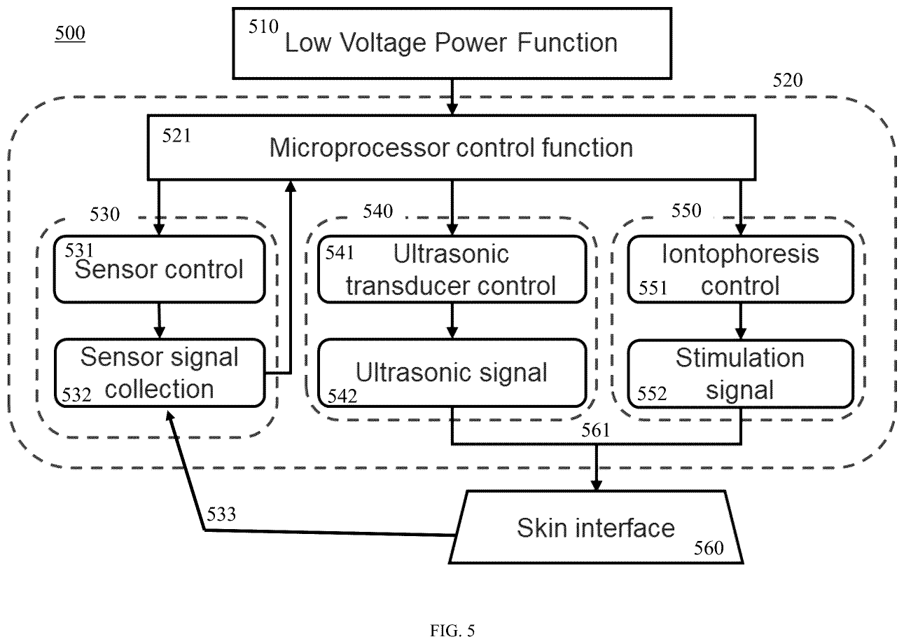

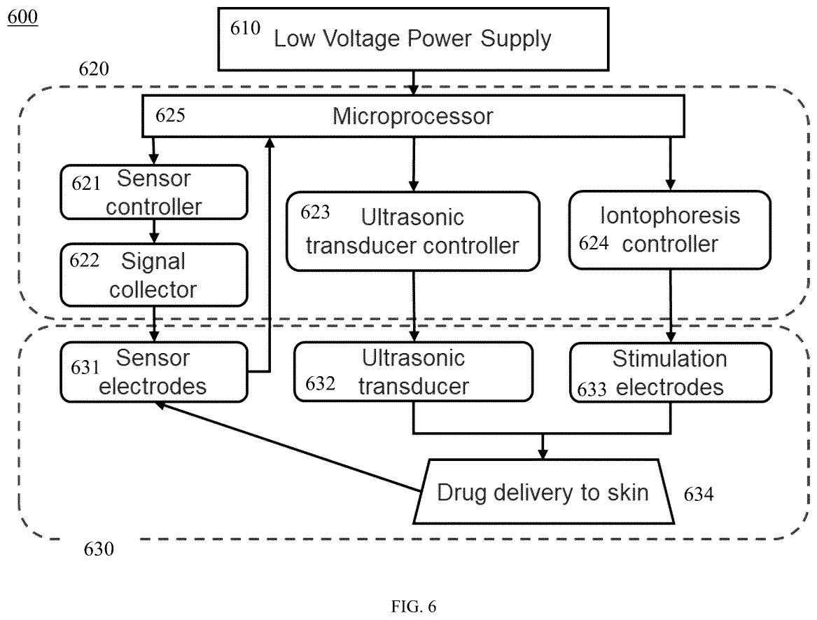

FIG. 5 illustrates a functional block diagram of a device (500) according to an embodiment of the subject invention. Low voltage power function (510) supplies power to support microprocessor control function (521), driving and controlling sensor function (530), ultrasonic function (540), and iontophoresis function (550). Sensor function (530) comprises sensor control (531) and sensor signal collection (532) to receive, process, and/or relay signals (533) from skin interface (560) to microprocessor control function (521). Ultrasonic function (540) comprises ultrasonic transducer control (541) and ultrasonic signal (542). Iontophoresis function (550) comprises iontophoresis control (551) and iontophoresis stimulation signal (552). Ultrasonic signal (542) and iontophoresis stimulation signal (552) can be delivered in combination (as shown) or separately (not shown) to skin interface (560) as skin signal (561). All functional elements described in relation to FIG. 5 can have directly analogous physical elements as shown in FIG. 5. Alternatively, functional elements described in relation to FIG. 5 can be supported by physical elements as shown in FIG. 6, or by other physical element configurations not shown.

FIG. 6 illustrates a physical assembly schematic diagram of a device (600) according to an embodiment of the subject invention. Low voltage power supply (610) is connected to control unit (620) comprising microprocessor (625), sensor controller (621), signal collector (622), ultrasonic generation controller (623), and iontophoresis controller (624), as shown. Stimulation unit (630) comprises sensor electrode(s) (631), ultrasonic transducer(s) (632), iontophoresis stimulation electrodes (633), and drug delivery to skin unit (634). Stimulation unit (630) can take the form of TDD device (100). Alternatively, stimulation unit (630) can take other forms.

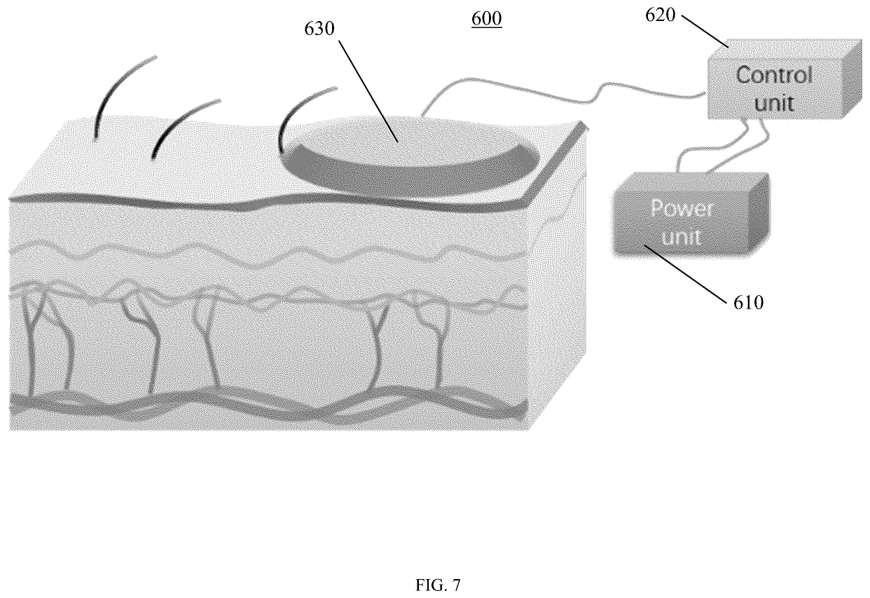

FIG. 7 illustrates a physical device (600) positioned on a dermal layer with a stimulation unit (630), a remote control unit (620) and a remote power supply unit (610) according to an embodiment of the subject invention. Alternatively, remote control unit (620) and remote power supply unit (610) can be combined in one physical unit (not shown). Alternatively, all three of stimulation unit (630), remote control unit (620), and remote power supply unit (610) can be combined in one physical unit (not shown). Alternatively, a multiplicity of stimulation units (630) can be driven by a single (e.g., remote or directly integrated) control unit (620), powered by a single (e.g., remote or directly integrated) power supply unit (610). Alternatively, a multiplicity of stimulation units (630) can be driven by a multiplicity (e.g., remote or directly integrated; independently autonomous, peer-to-peer-networked, or centrally coordinated and/or controlled) of control units (620), powered by one or more (e.g., remote or directly integrated) power supply unit(s) (610).

In the operation process according to certain embodiments of the drug delivery device, the workflow can be as follows. Firstly, the skin impedance measurement sensor can be used to continuously monitor the skin conditions (e.g., taking the skin impedance as an indicator of the permeability of the skin.) Upon a user's command or at a preset time, the iontophoresis controller and ultrasonic transducer controller can start operation and initiate the drug delivery process. In certain embodiments, if a drug contains small and ionic molecules, the device can be driven under electrical stimulation-only mode; on the other hand, if the drug molecules are large or neutral, both the ultrasonic and electrical stimulation can be turned on. The real-time value of the skin impedance can be used to determine the stimulation power of electrical and ultrasonic units to realize an appropriate drug delivery rate.

Transdermal drug delivery devices with a related art single stimulation modality have been proven to be useful for accelerating penetration of drug molecules into the skin [2]. Popular stimulation methods including electroporation, ultrasonic, electrical, thermal, microneedles, and other methods known in the art.

Regarding related art ultrasonic and electrical combined drug delivery devices, Kost et al., (U.S. patent application Ser. No. 10/839,571) describes an experimental observation that the combination of electric field stimulation and ultrasonic stimulation can promote penetration of drug molecules through the skin surface, however, it does not teach nor suggest any hardware design to realize the proposed electric field and ultrasonic stimulation. Moreover, the experiment was performed with and teaches only commercially available systems, which are large-size, heavy, bulky, and rigid, so that it cannot meet the requirements of wearable drug delivery devices (e.g., small-size, light weight, minimal bulk, flexible, and/or conformal). Embodiments of the subject invention provide (1) a novel and advantageous device structure design that allows for implementation of two stimulation units in a compact flexible and wearable device; and (2) integration of a sensing layer to provide feedback of the real-time skin condition for precise control of drug delivery.

Related art designs can combine an ultrasonic device and a pair of electrodes for drug delivery, for example, a structure in which the ultrasonic unit is placed above the electrode in Yang (U.S. patent application Ser. No. 15/752,693), or a structure in which the ultrasonic unit and the electrodes are arranged side by side in Jones, et. al (U.S. patent application Ser. No. 11/804,441.) However, these devices are bulky and cannot make close contact with the skin, nor can they provide real-time monitoring of the skin permeability. In contrast, embodiments of the subject invention are not only mechanically flexible (e.g., with the ability of bending to a radius as small as 10 mm), allowing for good contact with the skin, but also capable of real-time optimization of the stimulation power for good drug delivery results (advantageously enabled by the sensing layer and feedback controlling elements).

Clinical and commercial applications of transdermal drug delivery systems can include passive drug delivery technologies and active transdermal drug delivery. The transdermal drug delivery (TDD) devices can be used in a wide range of drug applications, including pain management, central nervous system disorders, hormonal applications, cardiovascular diseases, and others. Additionally, the TDD devices find utility in cosmetics applications such as acne treatments, skin hydration, anti-aging management, and other skincare solutions. Although currently TDD takes a small market share in the overall drug delivery market, it has recently gained market growth related to numerous advantages, including no first pass effect, reduction in pain, safer treatment, better biocompatibility, long-term and stable drug release, and others. With the advanced innovative technologies in the field of drug delivery, embodiments of the subject invention comprising active drug delivery systems can create a better solution for an aging global population and bring more convenience for patients.

All patents, patent applications, provisional applications, and publications referred to or cited herein are incorporated by reference in their entirety, including all figures and tables, to the extent they are not inconsistent with the explicit teachings of this specification.

Embodiments of the subject invention address the technical problem of controlling the application of feedback-controlled multi-modality stimulation to provide high efficiency and stable transdermal drug delivery being expensive, needing excessive human processing, not being suitable for wearable applications, and requiring costly, bulky, and complex equipment. This problem is addressed by providing a multilayered flexible transdermal drug delivery (TDD) system and/or methods of fabrication and operation thereof wherein a control unit providing operation and control of a multi-modality stimulation layer based on feedback from an adjacent or aligned skin sensing layer improves drug delivery in a compact, wearable, and flexible device. The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e., the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

### Materials and Methods

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

The following examples represent select prototype ultrasonic and electric embedded TDD devices with different closed-loop impedance sensors, fabricated and tested by the inventors according to embodiments of the subject invention. The fabrication process and material used have been developed and optimized. Both theoretical modelling and experimental results have confirmed the advantageous efficacy and practicality of the provided embedded TDD system.

### Example 1

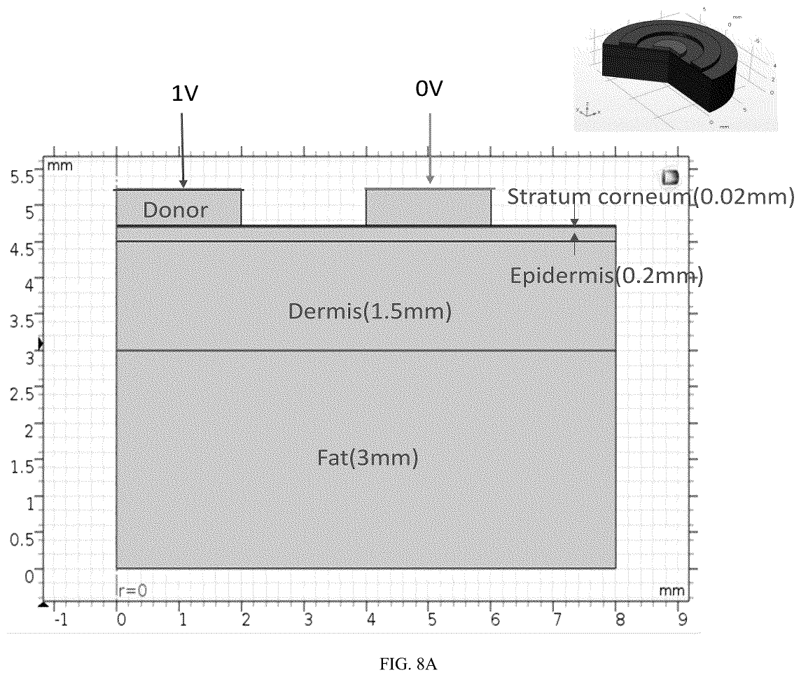

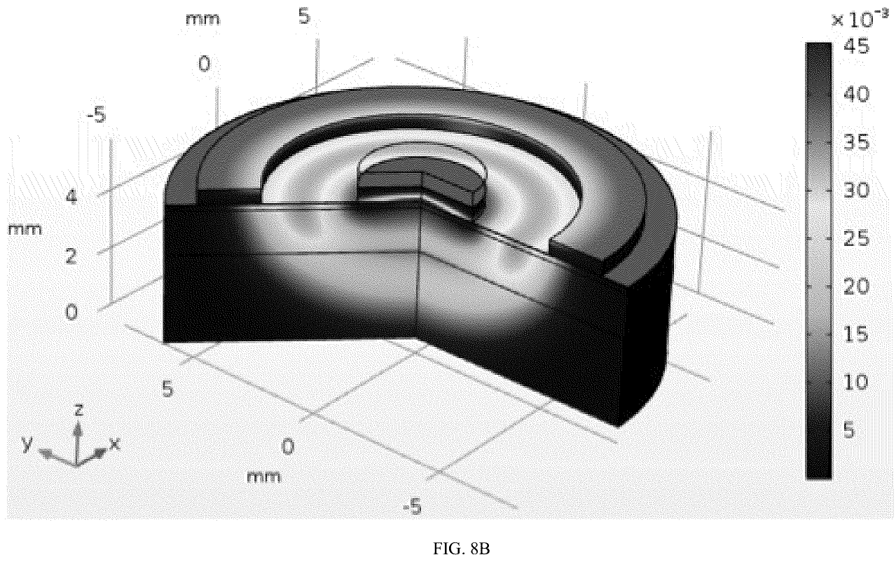

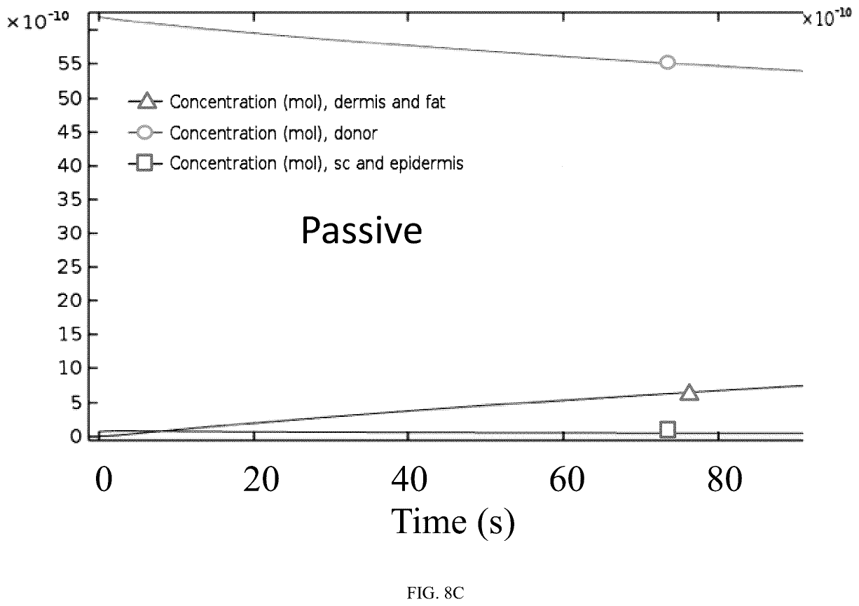

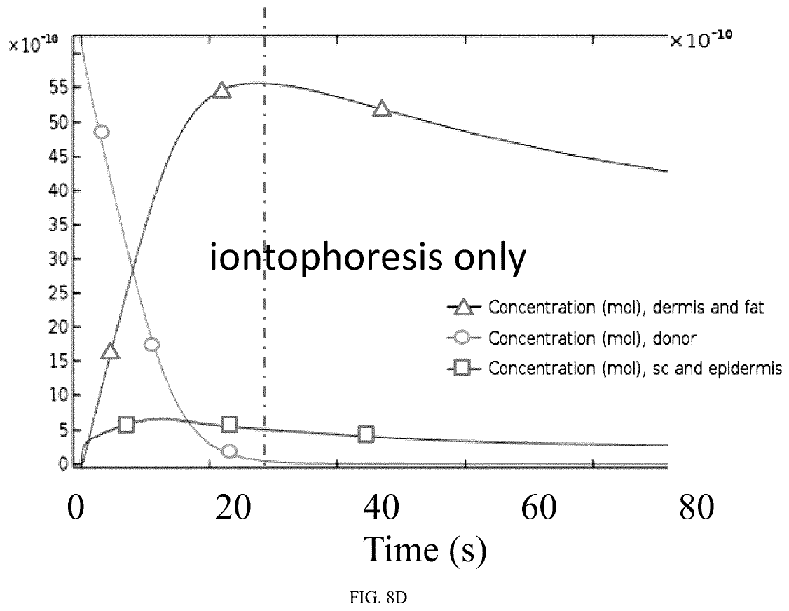

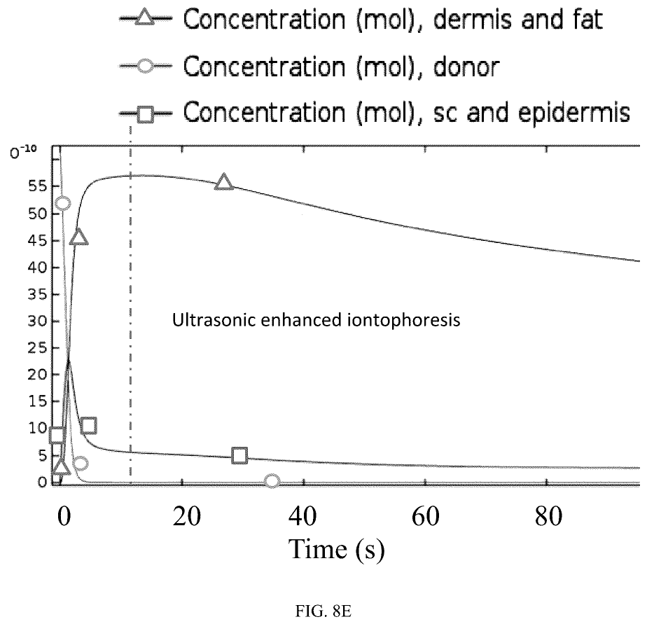

A finite element simulation model was established on COMSOL software to qualitatively illustrate the effect of the proposed multimodality stimulation on the drug penetration through the skin structure, as shown in FIG. 8A. The driving voltage of across the iontophoresis electrodes was set to 1 volt for passing the positively charged drug molecules. An ultrasonic generator with a characteristic frequency of 150 kHz and driving voltage amplitude of 2 V was applied above the iontophoresis electrode (FIG. 8B). To examine the iontophoresis/ultrasonic dual-modality effect, the inventors compared the drug penetration under three simulation conditions, i.e., (FIG. 8C) passive diffusion, (FIG. 8D) iontophoresis only, and (FIG. 8E) ultrasonic-enhanced iontophoresis. For passive diffusion the drug penetration volume peaks after 18000 seconds (predicted by the simulation but not shown in figure due to axis scale limit of the figure). In contrast, as shown FIGS. 8C, 8D, and 8E, this peaking time was shortened drastically to 27 seconds after iontophoresis and further down to 12 seconds with the enhancement of ultrasonic. Note that due to the limitation of the simulation, the skin barriers and steric hindrance of drug molecules are oversimplified. In reality, the skin has a more complex structure with stronger resistance and the drugs could be bulky and only weakly charged or neutral; as a result, the actual penetration rate under iontophoresis is hypothesized be much smaller than the simulation model predicted penetration rate.

### Example 2

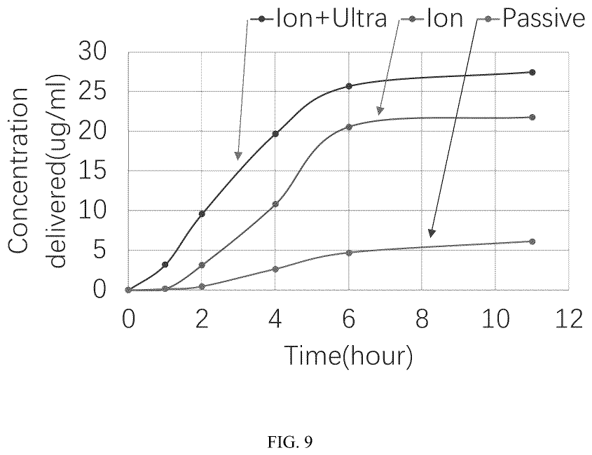

To experimentally validate the simulation result, we further performed an in vitro experiment with a Franz cell to compare the efficacy of iontophoresis/ultrasonic single-modality and dual-modality stimulation. The drug sample used is Sodium salicylate (NaC₆H₄(OH)CO₂) (160.11 g/mol), a common analgesic and antipyretic type drug, and its solution was deposited directly on the skin sample. In the passive process, no additional device is applied to the skin. In the “iontophoresis only” process, a pair of circular-shape iontophoresis electrodes made of PEDOT:PSS, mimicking the simulation model, were fabricated and placed on top of the skin, with the drug solution sandwiched between the narrow gaps between the electrodes and skin. For the “ultrasonic-enhanced iontophoresis” process, a disk-like ultrasonic device is placed on the PEDOT:PSS electrodes. As shown in FIG. 9, the drug penetration rate is enhanced by 4 and 5 times, respectively, by the “iontophoresis only” and “ultrasonic-enhanced iontophoresis” devices, confirming again that the dual-modality operation leads to the most efficiency drug penetration.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] Lu, F., Wang, C., Zhao, R., Du, L., Fang, Z., Guo, X., & Zhao, Z. (2018). Review of stratum corneum impedance measurement in non-invasive penetration application. Biosensors, 8(2), 31.
[2] Prausnitz, M. R., Mitragotri, S., & Langer, R. (2004). Current status and future potential of transdermal drug delivery. Nature reviews Drug discovery, 3 (2), 115-124.
[3] Kost, J., Pliquett, U., Mitragotri, S., Langer, R., & Weaver, J. (2004). U.S. patent application Ser. No. 10/839,571.
[4] Yang, Seong Seok. “Medical device for generating ultrasonic waves and electrical stimulation signal.” U.S. patent application Ser. No. 15/752,693.
[5] Jones, Dennis R., and Evan D. Parker. “Use of iontophoresis and ultrasonic to deliver melanin or other chromophores for laser hair removal.” U.S. patent application Ser. No. 11/804,441.
[6] BCC Research. (2021 July). Transdermal Drug Delivery: Global Markets (Report Code PHM254A). BCC Research. Retrieved May 23, 2022 from https://www.bccresearch.com/

1. A multilayered flexible transdermal drug delivery (TDD) system comprising:
a control unit (620) comprising:
a signal collector (622),
an iontophoresis controller (624), and
an ultrasonic generation controller (623);
a sensing layer (101) operably connected to the control unit (620), the sensing layer comprising:
a skin friendly contact layer (110), and
a sensor electrodes layer (120);
a multi-modality stimulation layer (105) physically connected directly on the sensing layer, the multi-modality stimulation layer comprising:
an electrical stimulation layer (102) operably connected to the control unit (620), the electrical stimulation layer (102) comprising:
an iontophoresis electrode layer (140), and
a substrate layer for electrodes (150); and
an ultrasonic stimulation layer (103) operably connected to the control unit (620) and physically connected directly on the electrical stimulation layer (102), the ultrasonic stimulation layer (103) comprising:
an ultrasonic generator layer (170), and
a bottom substrate layer with ultrasonic connectors (190).

2. The TDD system according to claim 1, comprising at least one additional functional layer selected from a heating layer, a cooling layer, a monitoring layer, a storage layer, a delivery layer, and a stimulating layer.

3. The TDD system according to claim 1, the sensing layer (101) configured and adapted to produce a skin impedance measurement.

4. The TDD system according to claim 3, the sensor electrodes layer (120) comprising three electrodes.

5. The TDD system according to claim 4, the three electrodes made from at least one material selected from silver (Ag), silver chloride (AgCl), a silver/silver chloride alloy (Ag/AgCl), another conductive metal, a conductive polymer, a conductive hydrogel, and a conductive non-metal, non-polymer material; and for conductive polymers, at least one of additives dopamine and (3-glycidyloxypropyl) trimethoxysilane (GOPS).

6. The TDD system according to claim 2, the control unit (620) configured and adapted to receive the skin impedance measurement from the sensing layer (101) through the signal collector and adjust a stimulation intensity through the iontophoresis controller to control an output through the iontophoresis electrode layer (140).

7. The TDD system according to claim 2, the control unit (620) configured and adapted to receive the skin impedance measurement from the sensing layer (101) through the signal collector and adjust a stimulation intensity through the ultrasonic controller to control an output through the ultrasonic generator layer (170).

8. The TDD system according to claim 5, the control unit (620) configured and adapted to receive the skin impedance measurement from the sensing layer (101) through the signal collector and adjust a stimulation intensity through the iontophoresis controller to control an output through the iontophoresis electrode layer (140).

9. The TDD system according to claim 8, the control unit (620) configured and adapted to receive the skin impedance measurement from the sensing layer (101) through the signal collector and adjust a stimulation intensity through the ultrasonic controller to control an output through the ultrasonic generator layer (170).

10. The TDD system according to claim 9,
the electrical stimulation layer (102) comprising:
a polymeric flexible layer (130) configured and adapted to deliver the drug through or around the sensing layer (101); and
the ultrasonic stimulation layer (103) comprising:
a top surface layer (160), and
a supplemental material layer (180).

11. The TDD system according to claim 10, the ultrasonic generator layer (170) comprising a solid disc type ultrasonic transducer with a solid disc electrode used as an ultrasonic stimulation generator.

12. The TDD system according to claim 10, the ultrasonic generator layer (170) comprising a ring type ultrasonic transducer with a ring electrode used as an ultrasonic stimulation generator.

13. The TDD system according to claim 10, the ultrasonic generator layer (170) comprising an ultrasonic transducer array with a corresponding number of stimulating electrodes and a common ground electrode configured as an ultrasonic stimulation generator.

14. The TDD system according to claim 10, the iontophoresis electrode layer (140) comprising any biocompatible ion conductive materials comprising at least one of poly(3,4-ethylenedioxythiophene): polystyrene sulfonate (PEDOT:PSS) and polyaniline (PANI).

15. The TDD system according to claim 10, the ultrasonic transducer layer (170) comprising at least one of Lead zirconate titanate (PZT) and Pb(Mg1/3Nb2/3)O3-PbTiO3 (PMN-PT).

16. A transdermal drug delivery (TDD) system for delivering a drug or cosmetic product to the skin of a patient, the TDD system comprising:
a sensing layer (101);
a multi-modality stimulation layer (105) physically connected on and aligned with the sensing layer (101), the multi-modality stimulation layer (105) comprising:
an electrical stimulation layer (102) comprising a polymeric flexible layer (130) configured and adapted to deliver the drug or cosmetic product through or around the sensing layer (101), and
an ultrasonic stimulation layer (103) physically connected on and aligned with the electrical stimulation layer (102); and
a control unit (620) operably connected to the sensing layer (101) and the multi-modality stimulation layer (105), the control unit (620) comprising:
a signal collector (622),
an iontophoresis controller (624),
an ultrasonic generation controller (623),
a processor (625) in operable communication with the signal collector (622), the iontophoresis controller (624), and the ultrasonic generation controller (623), and
a machine-readable medium in operable communication with the processor and having instructions stored thereon that, when executed by the processor, perform the following steps:
a) reading a first signal at a first time from the sensing layer (101),
b) activating the iontophoresis controller (624) at a first iontophoresis power level based on the first signal, and
c) activating the ultrasonic generation controller (623) at a first ultrasonic power level based on the first signal, to deliver the drug or cosmetic product to the skin of the patient.

17. The TDD system according to claim 16, the instructions, when executed by the processor, performing the following steps:
d) reading a second signal at a second time from the sensing layer (101),
e) activating the iontophoresis controller (624) at a second iontophoresis power level based on the second signal, and
f) activating the ultrasonic generation controller (623) at a second ultrasonic power level based on the second signal, to deliver the drug or cosmetic product to the skin of the patient.

18. The TDD system according to claim 17, the instructions, when executed by the processor, performing the following steps:
a) reading a multiplicity of signals over a period of time from the sensing layer (101),
b) actively controlling the iontophoresis controller (624) to a output a multiplicity of iontophoresis power levels, each respectively based on a respective signal of the multiplicity of signals, and
c) actively controlling the ultrasonic generation controller (623) to output a multiplicity of ultrasonic power levels, each respectively based on a respective signal of the multiplicity of signals, to deliver the drug or cosmetic product to the skin of the patient.

19. The TDD system according to claim 17, the instructions, when executed by the processor, performing the following steps:
a) reading a multiplicity of signals over a period of time from the sensing layer (101),
b) actively controlling the iontophoresis controller (624) to a output a multiplicity of iontophoresis power levels, each respectively based at least in part on the multiplicity of signals, and
c) actively controlling the ultrasonic generation controller (623) to output a multiplicity of ultrasonic power levels, each respectively based at least in part on the multiplicity of signals, to deliver the drug or cosmetic product to the skin of the patient.

20. The TDD system according to claim 18, the sensing layer (101) configured and adapted to produce a skin impedance measurement signal.

21. The TDD system according to claim 19, the sensor electrodes layer (120) comprising three electrodes made from at least one material selected from silver (Ag), silver chloride (AgCl), a silver/silver chloride alloy (Ag/AgCl), another conductive metal, a conductive polymer, a conductive hydrogel, and a conductive non-metal, non-polymer material, and for conductive polymers, at least one of additives dopamine and (3-glycidyloxypropyl) trimethoxysilane (GOPS).

22. The TDD system according to claim 16, wherein the cosmetic product contains hyaluronic acid or salicylic acid.

23. A multilayered flexible transdermal drug delivery (TDD) system comprising:
a control unit (620) comprising:
a signal collector (622),
an iontophoresis controller (624), and
an ultrasonic generation controller (623);
a sensing layer (101) operably connected to the control unit (620), the sensing layer comprising:
a skin friendly contact layer (110), and
a sensor electrodes layer (120);
a multi-modality stimulation layer (105) physically connected directly on the sensing layer, the multi-modality stimulation layer comprising:
an electrical stimulation layer (102) operably connected to the control unit (620), the electrical stimulation layer (102) comprising:
an iontophoresis electrode layer (140), and
a substrate layer for electrodes (150); and
an ultrasonic stimulation layer (103) operably connected to the control unit (620) and physically connected directly on the electrical stimulation layer (102), the ultrasonic stimulation layer (103) comprising:
an ultrasonic generator layer (170), and
a bottom substrate layer with ultrasonic connectors (190);
the sensing layer (101) configured and adapted to produce a skin impedance measurement;
the control unit (620) configured and adapted to receive the skin impedance measurement from the sensing layer (101) through the signal collector, adjust a stimulation intensity through the iontophoresis controller to control an output through the iontophoresis electrode layer (140), and adjust a stimulation intensity through the ultrasonic controller to control an output through the ultrasonic generator layer (170).

24. The multilayered flexible transdermal drug delivery (TDD) system according to claim 23, wherein:
the sensing layer (101) comprising three electrodes made from at least one material selected from silver (Ag), silver chloride (AgCl), a silver/silver chloride alloy (Ag/AgCl), another conductive metal, a conductive polymer, a conductive hydrogel, and a conductive non-metal, non-polymer material, and for conductive polymers, at least one of additives dopamine and (3-glycidyloxypropyl) trimethoxysilane (GOPS);
the electrical stimulation layer (102) comprising a polymeric flexible layer (130) configured and adapted to deliver the drug through or around the sensing layer (101);
the ultrasonic stimulation layer (103) comprising a top surface layer (160), and a supplemental material layer (180);
the ultrasonic generator layer (170) comprising either a solid disc type Lead zirconate titanate (PZT) transducer with a solid disc electrode used as an ultrasonic stimulation generator, or a ring type Lead zirconate titanate (PZT) transducer with a ring electrode used as an ultrasonic stimulation generator, or both.

25. A flexible drug delivery device with a multi-layer structure for creating a controllable and precise drug delivery process using ultrasonic and/or electrical stimulations to enhance or control drug penetration, the device comprising:
a sensing element that provides feedback on skin conditions and drug penetration process in the form of one or more sensing signals;
a stimulation unit comprising one or both of the following sub-units:
(1) a piezoelectric unit that generates ultrasonic stimulation, and
(2) a flexible electrode layer that produces electrical stimulation to skin;
a control unit that monitors the one or more sensing signals and controls the stimulation unit to modulate drug penetration; and
one or more flexible layers patterned with conductive electrodes that create an electrical connection from one or more of the sensing element, the stimulation unit, and the control unit to one or more external circuits.

26. An active, embedded, and layered transdermal drug delivery device comprising:
a stimulation layer, comprising at least one layer selected from the group consisting of:
an ultrasonic stimulation layer,
an electrical stimulation layer, and
a thermal stimulation layer;
a sensing layer comprising one or more surface impedance measurement electrodes configured and adapted to produce a skin impedance signal; and
a controller configured and adapted to provide a closed loop controlled drug release by driving the stimulation layer in response to the skin impedance signal.

27. A control system for a transdermal drug delivery device comprising:
an electric stimulation controller;
an ultrasonic wave generating and transmitting element;
an impedance measurement controller;
an ultrasonic generator controller; and
a central controller configured for generating and transmitting ultrasonic and electrical stimulations.
